(19) United States
(12) Patent Application Publication
(10) Pub. No.: US 2020/0032350 A1
(43) Pub. Date: Jan. 30, 2020

(54) COMPANION DIAGNOSTICS FOR LEUKEMIA TREATMENT

(71) Applicant: JOHANN WOLFGANG GOETHE-UNIVERSITÄT FRANKFURT AM MAIN, FRANKFURT AM MAIN (DE)

(72) Inventors: THOMAS OELLERICH, CAMBRIDGE (GB); HUBERT SERVE, FRANKFURT (DE); SEBASTIAN MOHR, FRANKFURT AM MAIN (DE); CARMEN DÖBELE, BÜRGSTADT (DE)

(21) Appl. No.: 16/499,290

(22) PCT Filed: Mar. 29, 2018
(86) PCT No.: PCT/EP2018/058220

§ 371 Date: Sep. 29, 2019

(30) Foreign Application Priority Data
Mar. 31, 2017 (EP) PCT/EP2017/057671

(51) Int. Cl. C12Q 1/6886; G16H 50/30; G16B 40/10

(52) U.S. Cl. CPC C12Q 1/6886; G16H 50/30; C12Q 2600/178; C12Q 2600/106; C12Q 2600/158; G16B 40/10

(57) ABSTRACT

The present invention pertains to a method for detecting whether a cancer disease in a subject is susceptible to a treatment with a Spleen Tyrosine Kinase (SYK) inhibitor by determining the amount or level of a biomarker selected from Hoxa9/Meis1, PU.1 and miR146a in a biological sample from the subject. The invention provides novel treatment approaches based on the detection of the differential expression of the above biomarkers using SYK inhibitors. Also provided are diagnostic kits, and combined therapeutic and diagnostic kits for use in the inventive methods.

### FIELD OF THE INVENTION

The present invention pertains to a method for detecting whether a cancer disease in a subject is susceptible to a treatment with a Spleen Tyrosine Kinase (SYK) inhibitor by determining the amount or level of a biomarker selected from Hoxa9/Meis1, PU.1 and miR146a in a biological sample from the subject. The invention provides novel treatment approaches based on the detection of the differential expression of the above biomarkers using SYK inhibitors. Also provided are diagnostic kits, and combined therapeutic and diagnostic kits for use in the inventive methods.

### DESCRIPTION

Acute myeloid leukemia (AML) is an aggressive neoplastic disease characterized by enhanced proliferation, blocked differentiation and dysregulated apoptosis. AML appears to be driven by cell populations exhibiting extensive self-renewal properties, known as leukemia stem cells (LSCs). Despite an increased understanding of the genetic mutations driving the development of AML, the molecular processes that govern these self-renewal properties remain elusive (Cancer Genome Atlas Research, 2013).

A large body of data implicates Hox genes in this process (Argiropoulos and Humphries, 2007). A central role for Hox genes in AML is supported by the frequently elevated Hox gene expression in AML cells (Afonja et al., 2000; Kawagoe et al., 1999; Lawrence et al., 1999). Hox gene overexpression is associated with genetically defined AML subgroups. Subsets of AML with favorable genetic features, such as core-binding factor leukemias and PML-RARα-positive leukemias, express low levels of Hox genes (Drabkin et al., 2002; Lawrence et al., 1999; Valk et al., 2004). In contrast, unfavorable genetic alterations, such as MLL fusions—for instance MLLAF9 and MLL-ENL—exhibit their transforming capacity largely through upregulation of Hox genes (Krivtsov and Armstrong, 2007; Muntean and Hess, 2012).

Among Hox genes, the Abd-B-type Hox genes (especially Hoxa9) are central regulators of the primitive hematopoietic compartment. Hoxa9 is preferentially expressed in primitive hematopoietic cells and is downregulated during differentiation (Pineault et al., 2002; Sauvageau et al., 1994). A number of overexpression studies have also shown that certain Hox genes and Hox gene fusions have the ability to promote expansion of primitive hematopoietic cells (Ohta et al., 2007; Sauvageau et al., 1995). Similarly, Hoxa9 enhances hematopoietic stem cell (HSC) regeneration in vivo, ultimately leading to the development of leukemia, albeit with a long latency (Thorsteinsdottir et al., 2002).

Meis1 is another critical regulator of LSCs that is often overexpressed in Hox-genedriven leukemia (Kawagoe et al., 1999; Lawrence et al., 1999). Although Meis1 alone is unable to promote self-renewal, it plays a role in establishing LSC potential in MLLrearranged leukemias (Wong et al., 2007). Moreover, when combined with overexpression of a Hox gene or the NUP98-Hox fusion gene, overexpression of Meis1 leads to a massive acceleration of leukemia development (Kroon et al., 1998; Pineault et al., 2004). Gene expression studies have identified a number of Meis1 target genes, some of which are critical for leukemogenesis (Argiropoulos et al., 2008; Kuchenbauer et al., 2011; Kuchenbauer et al., 2008; Wang et al., 2006). One such target is the tyrosine kinase Flt3, which in combination with a NUP98-Hox fusion gene accelerates leukemogenesis (Palmqvist et al., 2006; Wang et al., 2005). However, Flt3 appears to be dispensable for Meis1-induced leukemic transformation (Argiropoulos et al., 2008; Morgado et al., 2007).

While several studies have focused on Meis1 target genes, only a few have examined the intracellular signaling pathways affected by Meis1 overexpression. These studies showed that Meis1 enhances signaling through Akt and Erk (Argiropoulos et al., 2008) and activates the MAP kinase and PI3K/Akt pathways (Gibbs et al., 2012), and that activation of Wnt signaling is required for transformation of committed myeloid progenitors by Hoxa9 and Meis1 (Wang et al., 2010).

Because Hoxa9 and Meis1 overexpression is frequent in high-risk AML (Drabkin et al., 2002; Heuser et al., 2009; Zangenberg et al., 2009), and because both factors are currently consider-edundruggable, it was an object of the present invention to provide novel methods/means that allow clinicians to optimize cancer therapy in such situations.

The above problem is solved by a method for determining the sensitivity of a cancer patient for a Spleen Tyrosine Kinase (SYK) inhibitor therapy, the method comprising the steps of
(a) Determining in a biological sample of the patient the amount or level of
   (i) Meis1 and Hoxa9, and/or
   (ii) miR-146a, and/or
   (iii) PU.1
(b) Comparing the amount or level as determined in (a) with a reference or control, Wherein an increased amount or level compared to the reference or control of (i), or a reduced amount or level compared to the reference or control of (ii) and/or (iii), indicates sensitivity of the patient for a SYK inhibitor therapy.

The human Homeobox protein Meis1 is for example described in the UniProt database under the accession number O00470—release of Mar. 30, 2017. The Meis1 protein is encoded by the MEis1 gene (see HUGO Gene Nomenclature Committee accession number HGNC:7000—release of Mar. 30, 2017).

The human Homeobox protein Hox-A9 is for example described in the UniProt database under the accession number P31269—release of Mar. 30, 2017. The Hoxa9 protein is encoded by the HOXA9 gene (see HUGO Gene Nomenclature Committee accession number HGNC:5109—release of Mar. 30, 2017).

The human transcription factor PU.1 is for example described in the UniProt database under the accession number P17947—release of Mar. 30, 2017. The PU.1 protein is encoded by the SPI1 gene (see HUGO Gene Nomenclature Committee accession number HGNC:11241—release of Mar. 30, 2017).

The human miR-146a is derivable as hsa-miR-146a from miRBase.org (Accession number M10000477—release of Mar. 30, 2017).

In another aspect the invention provides a method for diagnosing a SYK inhibitor treatable cancer disease in a subject, the method comprising:
(a) Determining in a biological sample of the subject the amount or level of Meis1, or Meis1 and Hoxa9 expression,
(b) Comparing the amount or level of Meis1, or Meis1 and Hoxa9 expression, as determined in (a), with a control or reference,

Wherein an increased amount or level of Meis1, or Meis1 and Hoxa9 expression in the biological sample compared to the control or reference, indicates the presence of a SYK inhibitor treatable cancer disease in a subject.

In context of the present invention a “SYK inhibitor treatable cancer disease” is a cancer disease which is likely to respond to a SYK inhibitor treatment. A response is noted if progression of the cancer disease is reduced, halted or reversed.

The invention in another aspect pertains to a method for determining in a biological sample of a subject suffering from a cancer disease, the level of (i) Meis1 and Hoxa9, and/or (ii) miR-146a, and/or (iii) PU.1.

In another aspect the invention provides a method for diagnosing a SYK inhibitor treatable cancer disease in a subject, the method comprising:
(a) Determining in a biological sample of the subject the amount or level of PU.1 expression,
(b) Comparing the amount or level of PU.1 expression, as determined in (a), with a control or reference,

Wherein a decreased amount or level of PU.1 expression in the biological sample compared to the control or reference, indicates the presence of a SYK inhibitor treatable cancer disease in a subject.

In another aspect the invention provides a method for diagnosing a SYK inhibitor treatable cancer disease in a subject, the method comprising:
(a) Determining in a biological sample of the subject the amount or level of miR-146a expression,
(b) Comparing the amount or level of miR-146a expression, as determined in (a), with a control or reference,

Wherein a decreased amount or level of miR-146a expression in the biological sample compared to the control or reference, indicates the presence of a SYK inhibitor treatable cancer disease in a subject.

In context of the invention a cancer or a cancer disease is preferably a malignant proliferative disorder of any kind. Preferred cancers are selected from so called “liquid cancers”, such as lymphoma or leukemia, and preferably is acute myeloid leukemia (AML). Furthermore, the cancer in some embodiments is a Hox expression driven cancer, preferably a Hox expression driven AML, more preferably Hoxa9 driven AML.

The invention provides three biomarkers which were surprisingly associated with tumor cell addiction to SYK activity. Of these biomarkers in particular Meis1/Hoxa9 amount or level is determined in the mRNA level. The person of skill knows methods how to detect mRNA levels in biological samples of any kind. The detection of miR-146a is preferably also done using typical RNA detection methods. The detection of miRs in samples is also well known in the art. The PU.1 biomarker may be determined preferably immunological by detecting PU.1 protein in the biological sample of the subject. Alternatively it is possible to determine the PU.1 biomarker by detection of PU.1 mRNA expression.

In context of the invention mRNA expression and/or miR expression is determined by a method selected from PCR-based methods or in situ hybridization techniques, and/or wherein protein expression is determined immunological, for example by using an antibody based detection assay. Such assays involve in particular the use of nucleic acid based primers and/or probes comprising a sequence substantially identical to, or substantially complementary to, the gene sequence or mRNA or micro RNA sequence of the herein described biomarkers. Their respective gene or mRNA/micro RNA sequences are well known in the art and derivable from the respective public databases indicated above.

Preferably the PCR based methods of the invention involve the use of a primer or probe comprising a sequence complimentary to, or substantially complimentary to, a mRNA sequence of any of PU.1, Meis1 and/or HOXA9, and/or complimentary to, or substantially complimentary to the microRNA sequence of hsa-miR-146a.

Alternatively the detection of the biomarker of the invention as mentioned above may include the use of one or more antibodies specifically detecting a protein of PU.1, Meis1 and/or HOXA9. Such antibodies are available in the prior art or can be easily produced using standard antibody generation methods.

A biological sample of the patient in context of the invention is preferably a biological sample comprising at least one tumor cell of the cancer. Therefore, the present invention pertains to the determination of the biomarkers of the invention is a cancer (tumor) sample of the patient. Such a biological sample may be a tissue or liquid sample, preferably a blood sample or bone marrow sample. The person of skill understands that the nature of the biological sample suitable in the methods of the herein described invention will vary depending on the cancer disease. For example, tumor samples of solid tumors are mostly tissue samples, provided for example after tumor surgery or sampling. If the cancer disease is a cancer of the blood or bone marrow, tumor cells are often present in the blood stream of a patient. In this case the biological sample may be a blood sample or bone marrow sample.

In so far the methods of the invention are of a diagnostic nature, it may in some instances be preferred that the methods are strict in vitro or ex vivo methods. In other instances it is particular preferred that the methods comprise a step of obtaining the biological sample from the subject.

Since the invention seeks to provide better diagnostics and treatments for Hoxa9/Meis1 positive cancers, the patient benefitting from the invention is preferably a relapsed cancer patient and/or a cancer patient who received one or more non successful cancer therapies. Therefore, in some preferred embodiments the combined determination of the biomarkers of the invention is advantageous because the information retrieved provide the clinical practitioner with an indication which type of cancer treatment will likely be successful.

In another aspect the invention provides a method for the treatment of a Hoxa9/Meis1 positive cancer in a subject, the method comprising the administration of a therapeutically effective amount of a SYK inhibitor to the patient.

Furthermore provided is a method for treating a cancer disease in a patient, the method comprising the steps of
(a) Obtaining a biological sample from the patient,
(b) determining in the biological sample of the patient the level of (i) Meis1 and Hoxa9, and/or (ii) miR-146a, and/or (iii) PU.1,
(c) Administering to the patient a therapeutically effective amount of a cancer therapeutic,

wherein the cancer therapeutic administered to the patient in step (c) is selected from a SYK inhibitor if, in step (b), an increased amount or level compared to the reference or control of (i), or a reduced amount or level compared to the reference or control of (ii) and/or (iii), is determined in the biological sample of the patient; or wherein the cancer therapeutic administered to the patient in step (c) is selected from a compound other than a SYK inhibitor if no increased amount or level compared to the reference or control of (i), and no reduced amount or level compared to the reference or control of (ii) and/or (iii) is determined.

In context of the present invention a compound other than a SYK inhibitor shall be understood to broadly refer to any cancer treatment other than administration of a SYK inhibitor. Such treatments shall include any form of chemotherapy including the administration of any cytostatic or cytotoxic compounds indicated for treatment of the respective cancer disorder. In particular preferred are compounds used in therapy of leukemia. Further treatments include the use of radiotherapy or surgery.

In context of the present invention the term “subject” or “patient” preferably refers to a mammal, such as a mouse, rat, guinea pig, rabbit, cat, dog, monkey, or preferably a human, for example a human patient. The subject of the invention may be at danger of suffering from a proliferative disease such as a cancer or a tumor disease as described before, or suffer from a cancer or tumor disease as described before, preferably, wherein the tumor disease is a Meis1/Hoxa9 overexpressing cancer disease, such as AML.

In one embodiment, the SYK inhibitor of the invention is a modulator of expression, function and/or stability of SYK, or of a variant of SYK, may modulate SYK, or the variant of SYK, via a direct interaction (such as non-covalent and covalent binding) between the modulator and the SYK protein, or a protein of a SYK variant, their RNA transcripts or coding genomic loci. In other embodiments, the invention also includes modulators of SYK expression, function and/or stability that interact with one or more other components of the SYK-mediated immune modulatory mechanism and signaling pathway as disclosed herein and/or with one or more other genes that control the expression, function and/or stability of protein or mRNA of SYK, or of the variant of SYK. For example, a modulator of the invention may inhibit the expression, function and/or stability of SYK, or of a variant of SYK, binding directly to a protein of SYKo or the variant, and so for example inhibit the function of SYK or the variant (such as a modulator that is an inhibitory antibody against protein of SYK or of the variant), or may bind directly to mRNA of SYKo or the variant, and so for example inhibit the expression of SYK or the variant (such as a modulator that is an anti-sense nucleotide molecule against mRNA of SYK or of the variant). Alternatively, the modulator of the invention may inhibit the expression, function and/or stability of another gene that itself modulates the expression, function and/or stability of SYK, or of a variant of SYK; for example, a modulator that is an anti-sense nucleotide molecule against mRNA of an transcription factor for or repressor protein of SYK or for the variant. Mechanisms by which such modulation may be brought about, and/or the effects of such modulation, can include one or more of those as described elsewhere herein.

Particularly preferred modulators (in particular inhibitors/antagonists) of expression, function and/or stability of SYK, or of a variant of SYK, are in certain embodiments the following specific molecules and/or molecular classes. The modulator of expression, function and/or stability of SYK, or of a variant of SYK of the invention is in some embodiments selected from a compound which is polypeptide, peptide, glycoprotein, a peptide-mimetic, an antigen binding construct (for example, an anti-body, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA. The basic rules for the design of CRISPR/Cas9 mediated gene editing approaches are known to the skilled artisan and for example reviewed in Wiles M V et al.: “CRISPR-Cas9-mediated genome editing and guide RNA design.”, (Mamm Genome. 2015 October; 26(9-10):501-10) or in Savić N and Schwank G: “Advances in therapeutic CRISPR/Cas9 genome editing.” (Transl Res. 2016 February; 168:15-21).

Preferred SYK inhibitors of the invention are entospletinib, fostamatinib (R788), R406 or piceatannol.

Furthermore provided is a diagnostic kit for use in performing a method according to the invention, the kit comprising means for the detection of the amount or level of any of the biomarkers selected from Meis1, Hoxa9, miR-146a and PU.1.

In some embodiments of the invention the kit comprises means for the detection of the amount or level of Meis1 and Hoxa9, optionally for miR-146a and/or PU.1

In some embodiments of the invention the kit comprises means for the detection of the amount or level of miR-146a and/or PU.1.

The means are preferably any of the aforementioned primer/probes and/or antibodies suitable for use in the detection of the biomarkers of the invention as described before.

The diagnostic kit of the invention may in some embodiments also be a combined diagnostic/therapeutic kit. In this case the kit of the invention may further comprise a SYK inhibitor in an amount that when administrated to a cancer patient is effective for the treatment of the cancer.

The present invention will now be further described in the following examples with reference to the accompanying figures and sequences, nevertheless, without being limited thereto. For the purposes of the present invention, all references as cited herein are incorporated by reference in their entireties. In the Figures:

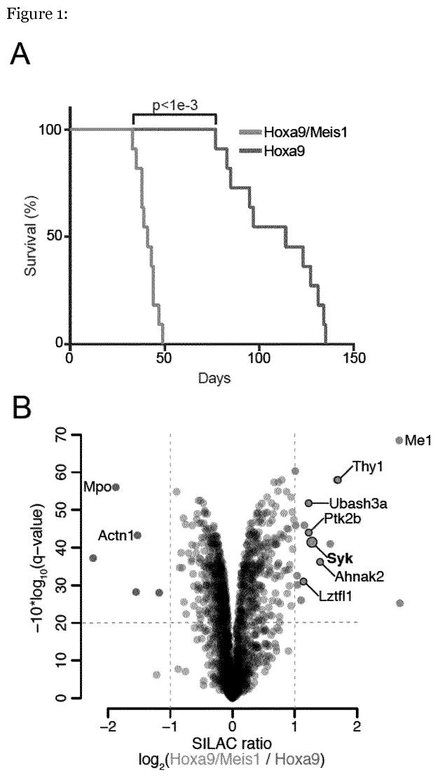

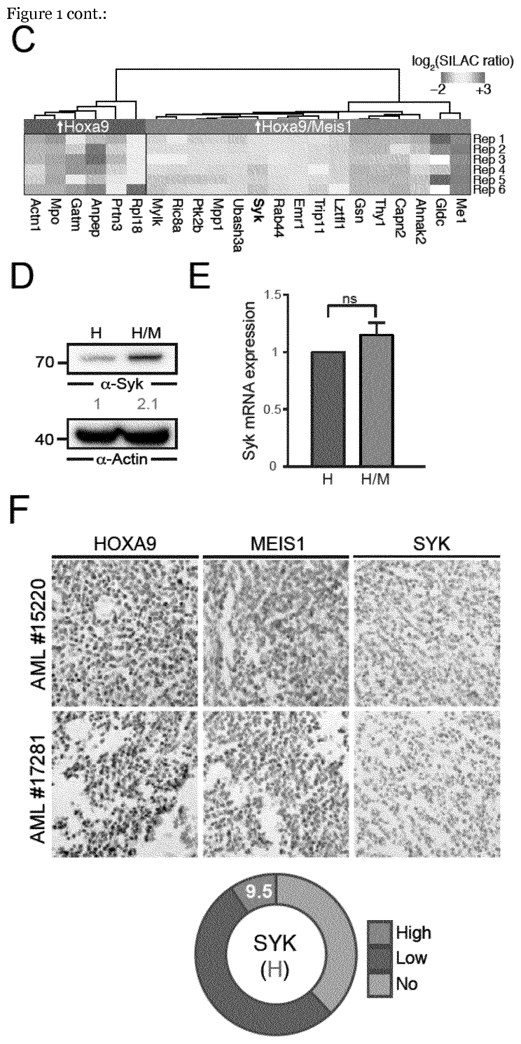

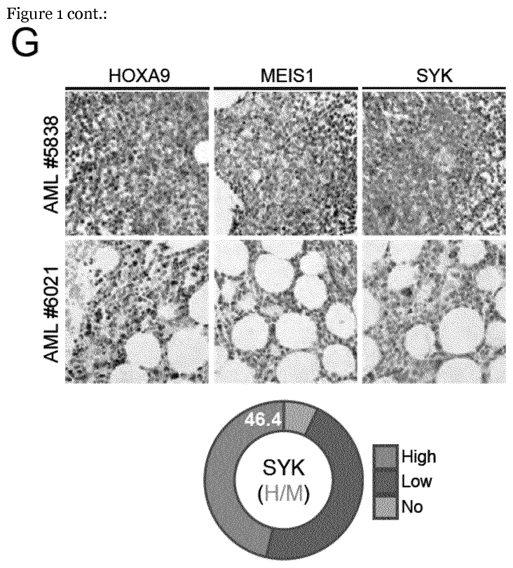

FIG. 1. Meis1 increases Syk protein levels in Hoxa9-driven leukemia. (A) Kaplan-Meier survival curves of mice transplanted with either H-or H/M-transformed myeloid progenitor cells (n=11). p-value is from a Mantel-Cox test. (B) Volcano plot relating q-values for differential protein expression to average normalized SILAC ratios from six biological replicates. Blue (higher expression in H cells) and orange (higher expression in H/M cells) dots indicate significantly regulated proteins (q<0.01). (C) Heatmap of SILAC ratios for significantly differentially expressed proteins in H and H/M cells across the six biological replicates. (D) Syk protein expression in H and H/M cells by immunoblotting. Actin was used as loading control for relative protein quantification. (E) Relative Syk mRNA expression as measured by qPCR, normalized to GAPDH expression (mean±SD, n=3); ns, not significant (twosided unpaired t-test) (F, G) Immunohistochemical staining (4ox magnification) of HOXA9, MEIS1 and SYK in bone marrow biopsies from patients with AML. SYK expression levels were analyzed in 21 AML cases with high HOXA9 expression (F) and 28 cases with high HOXA9/MEIS1 expression (G). Proportions of SYK expression levels as determined by two independent pathologists using a three-stage staining score are shown.

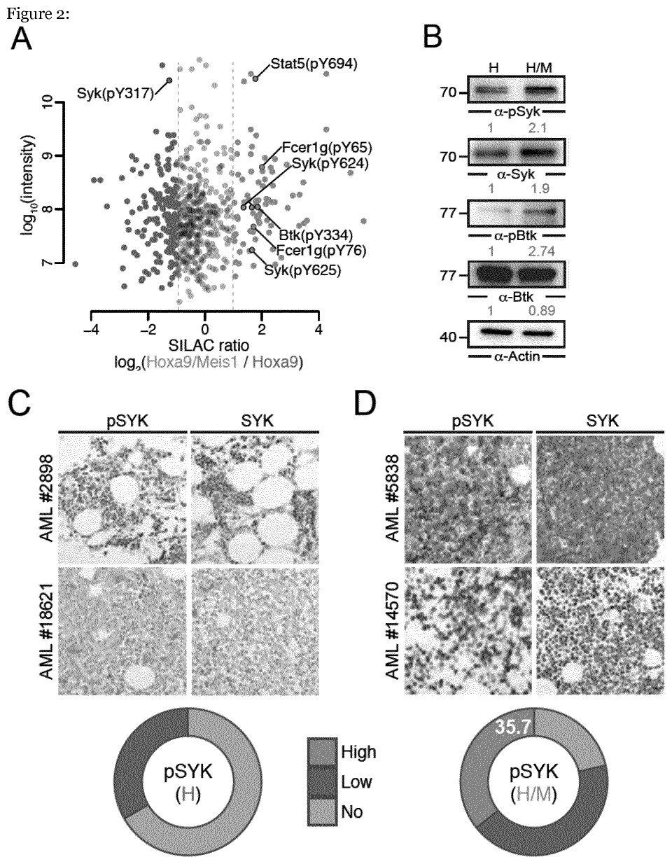

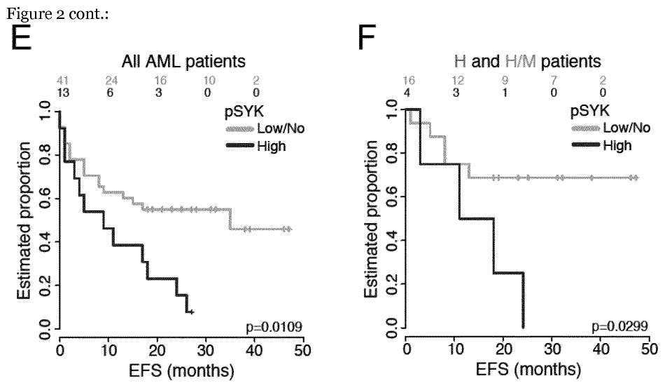

FIG. 2. Enhanced Syk signaling in H/M cells. (A) Intensities of peptide peaks versus average normalized SILAC ratios for p-sites identified by a massspectrometric pYome analysis in two biological replicates. Blue and orange dots indicate p-sites upregulated in H and H/M cells, respectively. Selected p-sites are labeled. (B) Validation of selected differential tyrosine phosphorylation events in H and H/M cells by immunoblotting. Actin was used as loading control for relative protein quantification. (C, D) Immunohistochemical staining (40× magnification) of phospho-SYK (pY348) and SYK in bone marrow biopsies from AML patients. pSYK levels were analyzed in 21 human AML cases with high HOXA9 expression (C) and 28 cases with high HOXA9/MEIS1 expression (D). Proportions of pSYK levels as determined by two independent pathologists using a three-stage staining score are shown. (E, F) Kaplan-Meier survival analysis for event-free survival (EFS) in which all AML patients with complete clinical profiles (E) or H and H/M patients only (F) were grouped by pSYK expression. The number of patients at risk belonging to each category is shown. p-value is from a Mantel-Cox test.

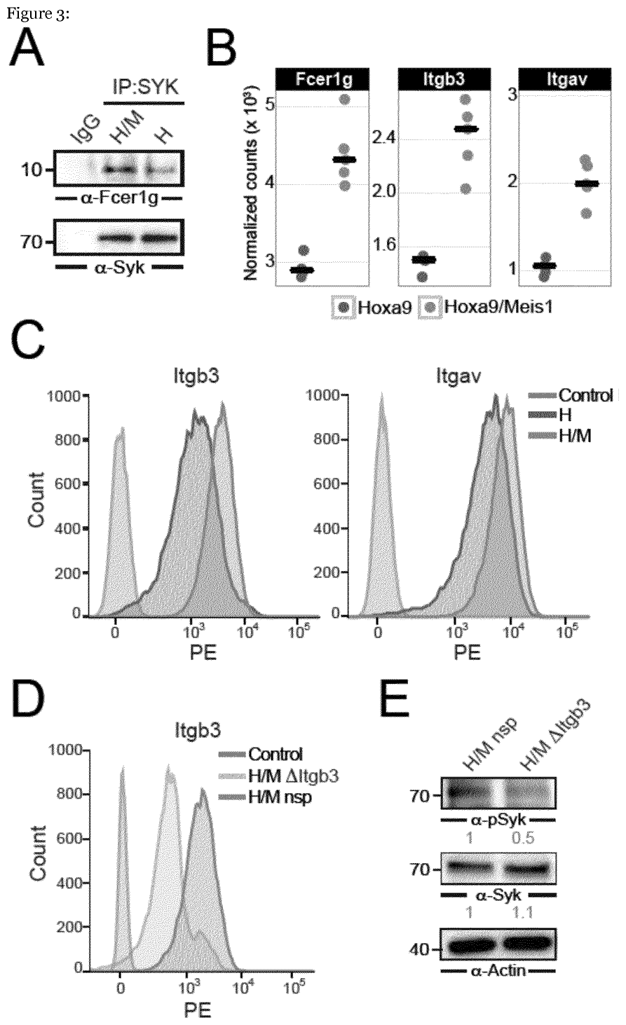

FIG. 3. Syk phosphorylation is partly dependent on integrin beta 3. (A) Coimmunoprecipitation of Fcer1g and Syk from H and H/M cells. (B) Fcer1g, Itgb3, Itgav expression estimated by normalized RNA-seq counts. (C) Itgb3 and Itgav cell surface expression in H and H/M cells measured by flow cytometry. Unstained cells were used as controls. (D) Itgb3 cell surface expression in H/M cells transduced with either a lentiviral non-specific (nsp) control CRISPR or a CRISPR targeting Itgb3 (ΔItgb3) (E) Corresponding (p)Syk expression determined by immunobloting. Actin was used as loading control for relative protein quantification.

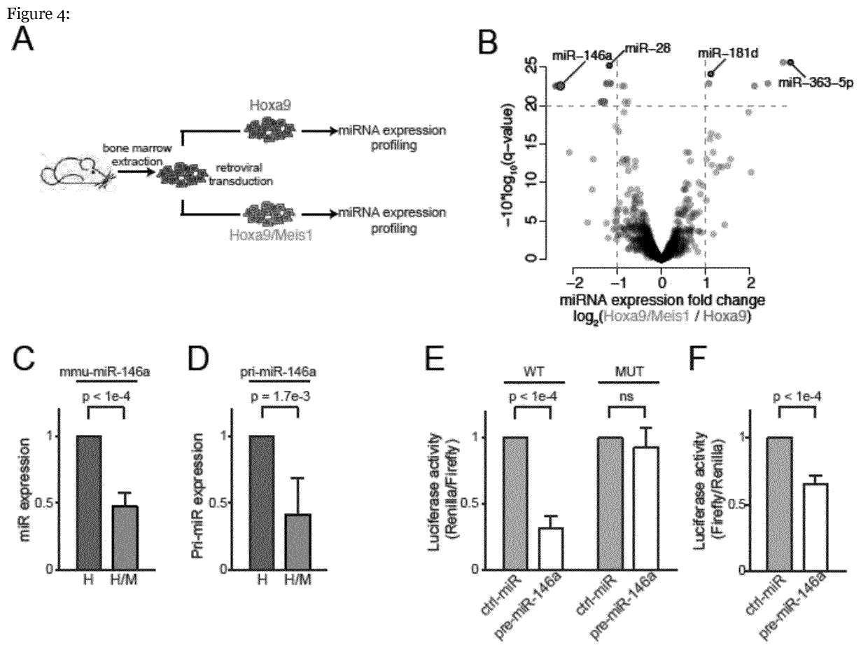

FIG. 4. Syk is a direct target of miR-146a. (A) Schematic workflow of the miRNA expression analysis in H- and H/M-transformed myeloid progenitors. (B) Volcano plot relating q-values for differential miRNA expression between H and H/M cells to average miRNA expression fold changes from three biological replicates. Blue (higher expression in H cells) and orange (higher expression in H/M cells) dots indicate significantly regulated miRNAs (q<0.01). (C, D) Relative mmu-miR-146a expression (C) and pri-miR-146a expression (D) in H/M versus H cells, measured by qPCR and normalized to sno202 and GAPDH expression, respectively (mean±SD, n=3). p-values are from two-sided unpaired t-test. (E) Luciferase assay validating binding of miR-146a to the predicted target sites within the 3′-UTR of Syk (mean±SD, n=4); WT, predicted miR-146a target sequence; MUT, mutated version thereof. pvalues are from two-sided unpaired t-test. ns, not significant. (F) Luciferase assay validating binding of miR-146a to the full-length Syk 3′-UTR (mean±SD, n=4). pvalue is from two-sided unpaired t-test. (G) (left) Secondary structure of mmu-miR146 as predicted by RNAfold (Lorenz et al., 2011). The CRISPR/Cas9 cleavage site is indicated. (right) Relative expression of miR-146a, measured by qPCR and normalized to sno202 expression, in H cells transduced with either a lentiviral non-specific (nsp) control CRISPR or a CRISPR targeting miR-146 (ΔmiR-146) (mean±SD, n=3). p-value is from two-sided unpaired t-test. (H) Corresponding Syk protein expression by immunoblotting. Actin was used as loading control for relative protein quantification. (I) Cell proliferation curves for H cells transduced with either a lentiviral non-specific (nsp) control CRISPR or a CRISPR targeting miR-146 (ΔmiR146) (mean±SD, n=3). (J) Kaplan-Meier survival curves of mice transplanted with H or H/M cells transduced with a lentiviral non-specific (nsp) control CRISPR, or with H cells transduced with a CRISPR targeting miR-146 (ΔmiR-146) (n=7). p-value is from a Mantel-Cox test.

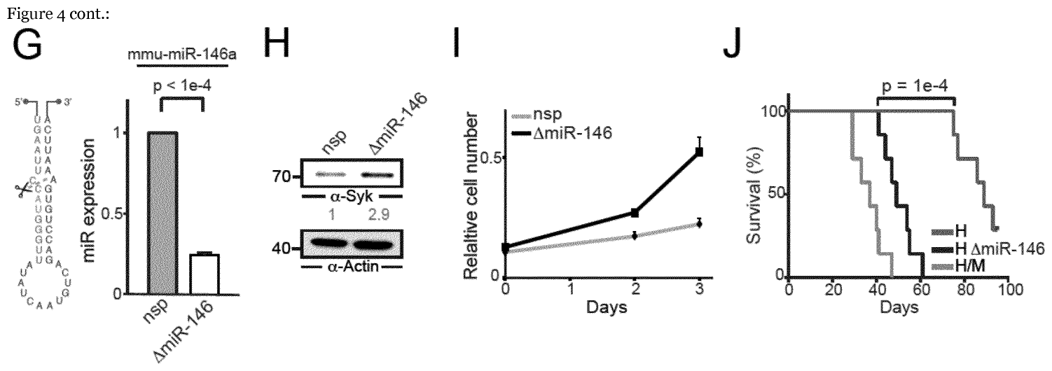

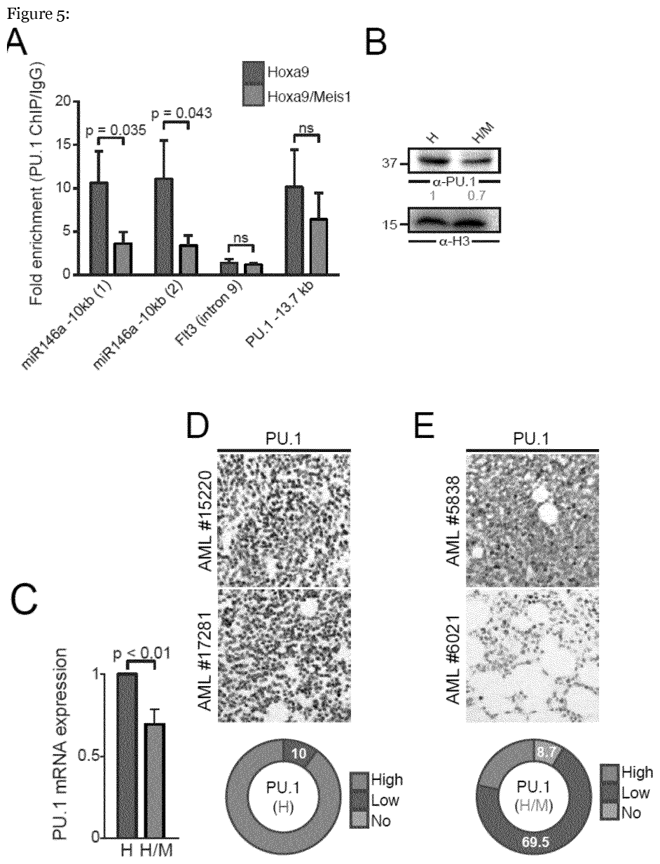

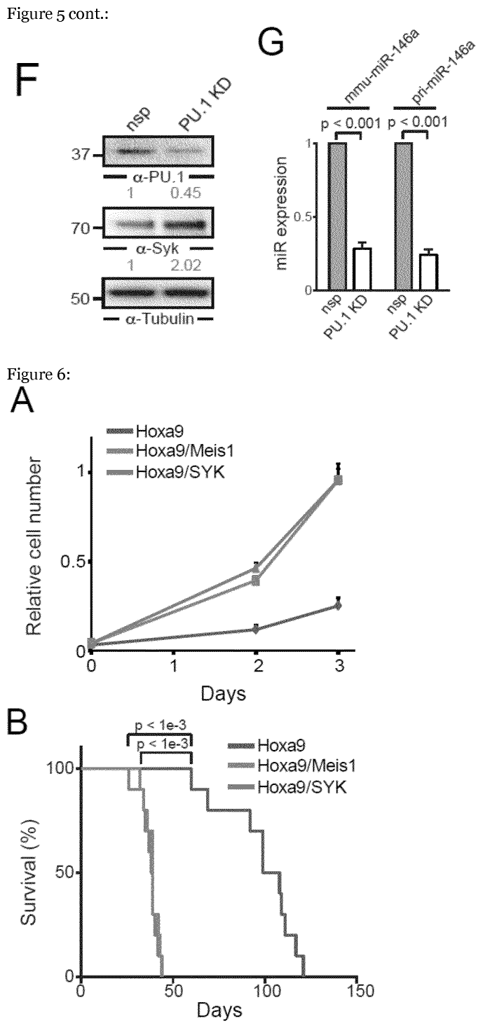

FIG. 5. Meis1 downregulates miR-146a through PU.1. (A) Fold enrichment of PU.1 binding over IgG control as measured by ChIP-qPCR in H and H/M cells (mean±SD, n=3). The miR-146a −10 kb region spans the transcription start site of the miR-146a host gene; ns, not significant. (B) PU.1 protein expression in H and H/M cells by immunoblotting. Histone H3 was used as loading control for relative protein quantification. (C) Relative PU.1 mRNA expression in H versus H/M cells measured by qPCR and normalized to GAPDH expression (mean±SD, n=3). (D, E) Immunohistochemical staining (4ox magnification) of PU.1 in bone marrow biopsies from patients with AML. PU.1 expression levels were analyzed in 21 AML cases with high HOXA9 expression (D) and 28 cases with high HOXA9/MEIS1 expression (E). Proportions of PU.1 expression levels as determined by two independent pathologists using a three-stage staining score are shown. (F) PU.1 and SYK protein expression by immunoblotting in H cells transfected with either a control shRNA (nsp) or an shRNA targeting PU.1 (KD). Tubulin was used as loading control for relative protein quantification. (G) mmu-miR-146a and pri-miR-146a expression as measured by qPCR after PU.1 knockdown (KD) relative to control shRNA (nsp) (mean±SD, n=4). p-values are from two-sided unpaired t-test.

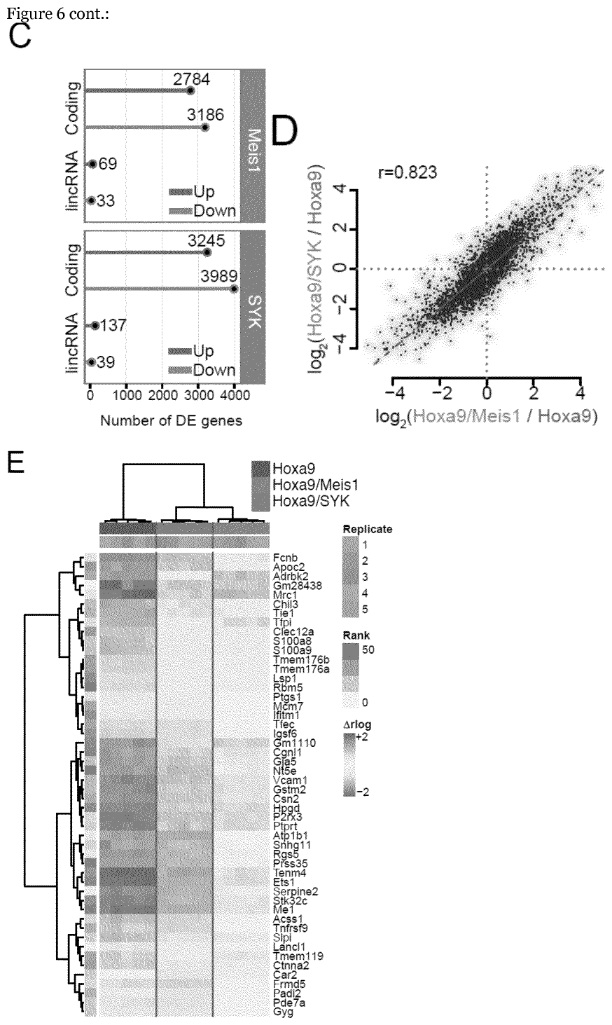

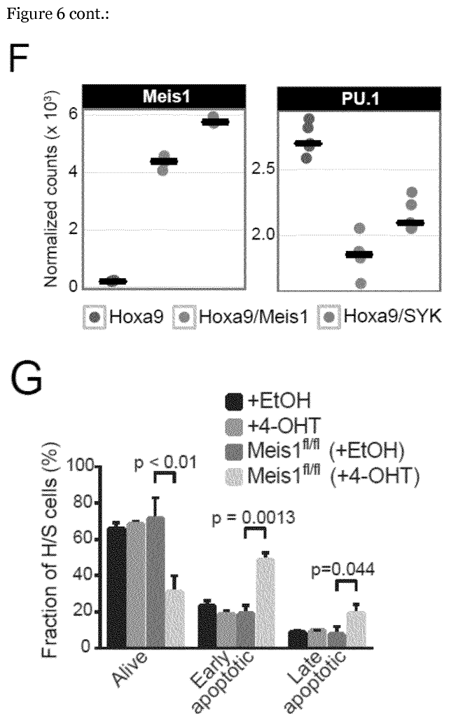

FIG. 6. Syk overexpression mimics the leukemogenic Meis1 transcriptional program in Hoxa9-driven leukemia. (A) Proliferation curves for H, H/M and H/S cells (mean±SD). (B) Kaplan-Meier survival curves of mice transplanted with either H (n=9), H/M (n=10) or H/S (n=11) cells. p-values are from a Mantel-Cox test. (C) Summary of differentially expressed (DE) protein-coding genes and lincRNAs (BH adjusted p-value 0.001, Wald test) in H-transformed myeloid progenitors upon overexpression of Meis1 (upper panel) and SYK (lower panel). (D) Gene expression correlation between H/M and H/S cells. Only genes that were significantly differentially expressed in at least one condition (BH adjusted p-value ≤10-5, likelihood ratio test) were considered. Correlation value (r) is Spearman's rank correlation coefficient. (E) Hierarchical clustering of the top 50 differentially expressed genes. (F) Meis1 and PU.1 expression estimated by normalized RNA-seq counts. (G) Apoptosis analysis of H/S cells derived from either C57BL/6J mice or inducible Meis1 knockout mice, based on Annexin V/7-AAD staining (mean±SD, n=3). Cells were treated with either ethanol (EtOH, control) or 4-hydroxytamoxifen (4OHT). p-values are from two-sided unpaired t-test.

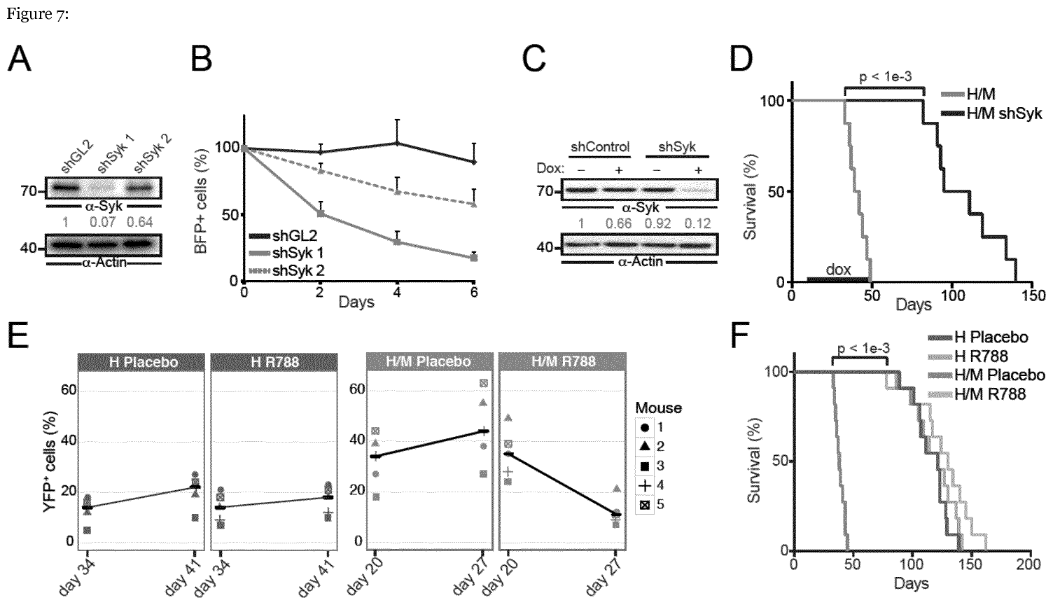

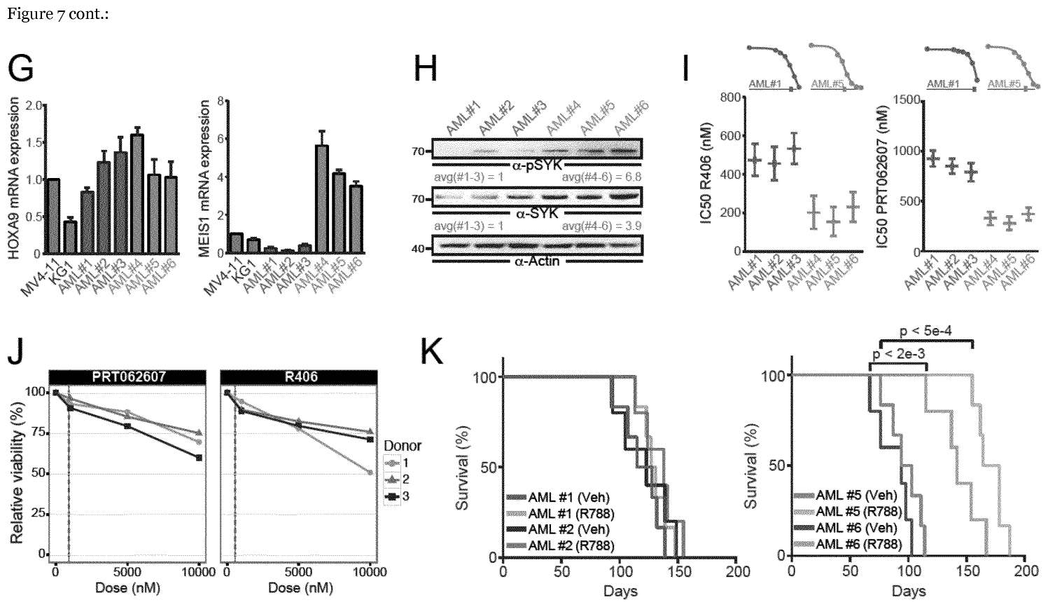

FIG. 7. Meis1 sensitizes Hoxa9-driven leukemia to Syk inhibition. (A) SYK protein expression in H/M cells transfected with either a control shRNA (GL2) or two shRNAs targeting SYK. Actin was used as loading control for relative protein quantification. (B) Percentage of BFP-positive shRNA-expressing cells relative to BFP-negative shRNA-negative cells at the times indicated (mean±SD, normalized to day 0). (C) Same as (A), before and after five days of doxycycline (dox) treatment in vivo. (D) Kaplan-Meier survival curves of mice transplanted with H/M cells and treated with doxycycline for 43 days to express non-specific control and Syk-specific shRNA (n=8). p-value is from a Mantel-Cox test. (E) Percentage of YFP-positive cells from peripheral blood of mice transplanted with H (left) or H/M (right) cells after treating for 7 days with R788 or placebo. Measurements were taken at the indicated time points. The black line connects median values. (F) Kaplan-Meier survival curves of mice transplanted with either H or H/M cells and treated for 20 days with R788 or placebo (n=11). p-value is from a Mantel-Cox test. (G) Relative HOXA9 and MEIS1 mRNA expression in MV4-11 and KG1 cell lines, and in patient-derived AML cells as measured by qPCR, normalized to GAPDH expression (mean±SD, n=3). (H) (p)SYK expression in the patient-derived AML cells in (G). Actin was used as loading control for relative protein quantification. avg, average. (I) IC50 for R406 and PRT062607 in patient-derived AML cells as determined by a Annexin V/7-AAD apoptosis assay. Cells were treated for 24 h and DMSO was used as a control. (J) Relative viability of CD34+ bone marrow cells from healthy donors. Cells were treated with either R406 or PRT062607. Blue lines indicate the IC50 for both SYK inhibitors in H cells. (K) Kaplan-Meier survival curves of NSG mice transplanted with patient-derived AML cells indicated in (G) and treated for 14 days with R788 or vehicle (n=6 for AML #1 and #5; n=5 for AML #2 and #6). p-values are from a Mantel-Cox test.

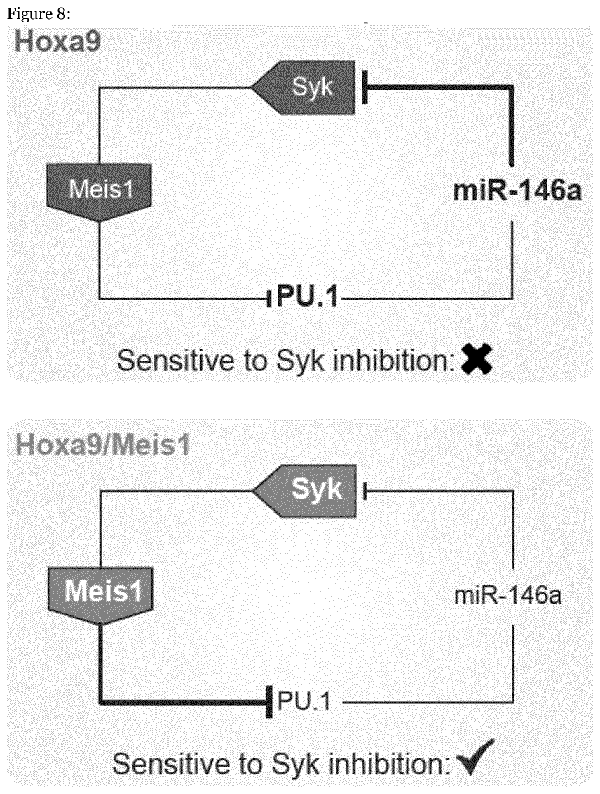

FIG. 8: shows the interrelation of the HoxA9/Meis1/SYK system

### EXAMPLES

Example 1: Meis1 Induces Syk Signaling in Hoxa9-Overexpressing Myeloid Progenitors

To elucidate the molecular mechanisms underlying Meis1's contribution to leukemogenesis, a retroviral transplantation model was employed in which lineage-depleted mouse bone marrow cells were transduced with an MSCV-Hoxa9-PGK-neo construct, alone or in combination with an MSCV-Meis1-IRES-YFP construct that induced a 22-fold overexpression of Meis1. As reported by previously, the transformed cells could be cultured in vitro in the presence of IL3/IL6/SCF and expressed the expected immunophenotype characterized by the myeloid markers Mac-1 and Gr-1 as well as c-Kit (Pineault et al., 2005; Wang et al., 2005). When transplanted into irradiated recipient mice, cells transduced with Hoxa9 (H) or Hoxa9/Meis1 (H/M) gave rise to leukemia resulting in a median overall survival of 114 days and 41 days, respectively (p<0.001; FIG. 1A). This difference in survival is in accordance with previously published results and reflects the aggressiveness of Hoxa9/Meis1-driven AML observed in patients (Kroon et al., 1998).

Because mRNA expression levels only moderately correlate with actual protein levels (Schwanhausser et al., 2011; Vogel and Marcotte, 2012), the consequences of Meis1 expression on the cellular proteome was analyzed by combining stable isotope labeling by amino acids in cell culture (SILAC) and mass spectrometry. This quantitative protein expression analysis of H and H/M cells was performed in six biological replicates and allowed the reproducible identification and quantification of 1810 proteins in at least four out of six biological replicates.

Interestingly, two tyrosine kinases, focal adhesion kinase 2 (Ptk2b) and spleen tyrosine kinase (Syk), were among the most upregulated proteins in H/M cells (FIGS. 1B and 1C). Overexpression of Syk in H/M cells was confirmed by immunoblotting (FIG. 1D). Notably, quantitative real-time polymerase chain reaction (qPCR) and RNA sequencing (RNA-seq) indicated that Syk was not upregulated at the mRNA level in H/M cells (qPCR fold change 1.15; RNA-seq fold change 1.13; FIG. 1E), thus explaining why several independent RNA expression analyses did not link Syk to Meis1 (Argiropoulos et al., 2008; Argiropoulos et al., 2010; Huang et al., 2012; Wang et al., 2005; Wang et al., 2006; Wilhelm et al., 2011). To test whether the combined H/M overexpression is also associated with enhanced Syk protein expression in primary human AML samples, the inventors performed immunohistochemical (IHC) analyses for HOXA9, MEIS1 and SYK on a cohort of 115 AML cases. Overexpression of HOXA9 alone in a total of 21 cases and overexpression of both HOXA9 and MEIS1 in 28 cases was found, with only one Flt3-ITD positive patient. Increased SYK expression was significantly more frequent (>4 times, p=0.01, Fisher's exact test) in samples with a high expression of HOXA9 and MEIS1 (46.4%) than in HOXA9-overexpressing samples (9.5%) (FIGS. 1F and 1G). This frequency is also >2 times higher than in HOXA9 and MEIS1 double negative samples (22.7%). Hence, combined overexpression of Hoxa9 and Meis1 leads to upregulation of Syk at the post-transcriptional level, and elevated SYK expression is associated with HOXA9/MEIS1 overexpression in human AML samples.

The deregulated expression of kinases prompted the inventors to examine the global impact of Meis1 overexpression on intracellular signaling by a mass-spectrometry-based phosphoproteomic analysis of H and H/M cells. The analysis was performed after enrichment for phosphorylated tyrosine residues (pYome) and separately after enrichment for phospho-serine, -threonine and -tyrosine residues (Global phosphoproteome, GPome). The inventors identified and quantified a total of 584 class-I phosphorylation events (p-events with a localization probability >75%) in the pYome and 3305 class-I p-events in the GPome, of which 236 and 297 were differentially regulated between H and H/M cells, respectively (FIG. 2A). Notably, this analysis revealed enhanced phosphorylation of the Syk-activating tyrosines Y624/625 and dephosphorylation of the inhibitory tyrosine Y317 in H/M cells, suggesting enhanced Syk signaling in H/M cells. This result is furthermore supported by an enhanced tyrosine-phosphorylation of STATS and BTK, two effectors known to be activated by Syk in AML and B-cells, respectively (Carnevale et al., 2013; Oellerich et al., 2013) (FIG. 2A). Differential phosphorylation of Syk and Btk was confirmed by immunoblotting (FIG. 2B).

As Meis1 not only enhanced Syk protein expression, but also increased its activation-inducing tyrosine-phosphorylation in this model system, this finding was validated in a cohort of primary AML samples. Therefore, IHC analyses for phosphorylated SYK (pY348, a Syk-activating p-site) was performed in the 21 AML cases overexpressing HOXA9 alone and in the 28 cases overexpressing both HOXA9 and MEIS1 (FIGS. 2C and 2D). This analysis revealed a significant association between strong SYK phosphorylation and HOXA9/MEIS1 overexpression (35.7% of H/M samples) compared with samples in which only HOXA9 was overexpressed (0% of H samples; p<0.003, two-sided Fisher's exact test) or double negative samples (13.6%; p=0.024) (FIGS. 2C, 2D and S2C). Moreover, high SYK phosphorylation correlates with poor event-free and relapse-free survival in the subset of AML patients with complete clinical profiles within the cohort, both with and without stratification for H and H/M expression (FIGS. 2E, 2F). Together, these results indicate a strong association of MEIS1 overexpression with upregulation and activation of SYK in AML.

Example 2: Enhanced Syk Activation is Partly Dependent on Integrin Beta 3

Next, the potential mechanisms of Syk activation in H/M cells were investigated. Syk activation requires docking to phosphorylated immunoreceptor tyrosine-based activation motifs (ITAMs) (Kulathu et al., 2009). Interestingly, the pYome analysis revealed increased ITAM phosphorylation of the common Fey-chain Fcer1g in H/M cells (FIG. 2A). Fcer1g is an intracellular signaling module that associates with Fc receptors and integrins (Humphrey et al., 2005). While depletion of Fc receptors does not affect viability and proliferation of AML cells, integrin beta 3 (Itgb3) is required for leukemogenesis (Miller et al., 2013; Oellerich et al., 2013). Notably, Fcer1g interacts with Syk in H cells, and in line with enhanced ITAM phosphorylation, this interaction is stronger in H/M cells (FIG. 3A). In addition, Meis1 overexpression in H cells increased transcript levels of Fcer1g, Itgb3 and its heterodimeric partner integrin alpha v (Itgav), and upregulated Itgb3/Itgav expression on the cell surface (FIGS. 3B and 3C). To test whether increased Itgb3 cell surface expression translates into increased Syk activity, the inventors knocked out Itgb3 using CRISPR/Cas9 by transducing H/M cells with a lentiviral Itgb3 CRISPR construct (ΔItgb3) (FIG. 3D). Itgb3 knockout led to a 50% reduction in activatory Syk phosphorylation (pY525/526) in a polyclonal cell population (FIG. 3E), indicating that enhanced Syk activation in H/M cells depends—at least in part—on integrin beta 3.

Example 3: Syk Expression is Regulated by miR-146a

Because the upregulation of Syk in H/M cells was only detectable at the protein but not at the mRNA level (FIGS. 1B-E), and because no differences were detected in the proteasomal degradation of Syk (data not shown), it was reasoned that miRNA(s) might be involved in the regulation of Syk. To test this hypothesis, miRNA expression was globally profiled in H and H/M cells (FIG. 4A). This analysis identified eight significantly downregulated miRNAs in H/M cells potentially responsible for the observed upregulation of Syk (FIG. 4B). To refine the candidate list, only those miRNAs were retained that were predicted to target Syk by Targetscan (Agarwal et al., 2015). The algorithm identified two predicted binding sites for miR146a in the 3′-UTR of Syk. A significant downregulation of mmu-miR-146a and primiR-146a in H/M relative to H cells was further confirmed by qPCR (FIGS. 4C and 4D).

To experimentally validate targeting of Syk by miR-146a, luciferase assays were performed using two reporter constructs, one containing two copies of both predicted miR-146a binding sites (or mutated versions as controls; FIG. 4E) and one containing the full-length Syk 3′-UTR (FIG. 4F). Overexpression of miR-146a precursor (pre-miR-146a) decreased luciferase activity in lysates of HEK293T cells transfected with the construct containing the miR-146a target sites or the Syk 3′UTR, but had no effect on the construct with mutated binding sites (FIGS. 4E and 4F). This result indicates that Syk is a direct miR-146a target.

To further test whether miR-146a affects Syk expression, we knocked out miR-146 using CRISPR/Cas9 by transducing H cells with a lentiviral miR-146-specific CRISPR construct (ΔmiR-146) that reduced miR-146a expression by 75% in a polyclonal cell population (FIG. 4G) or isolated myeloid progenitor cells from B6/miR-146a^{-/-} mice and transduced them with Hoxa9. The CRISPR-mediated knockout of miR-146 led to a 2.9-fold increase in the protein expression of Syk (FIG. 4H), increased cell proliferation (FIGS. 41 and S3B), reduced apoptosis (FIG. S3C) and enhanced c-Kit expression (FIG. S3D), mirroring the phenotype of H/M cells. Finally, mice transplanted with miR-146 knockout H cells exhibited accelerated leukemia development compared to mice transplanted with H cells (FIG. 4J). In summary, these data strongly indicate that upregulation of Syk in H/M cells is mediated by downregulation of miR-146a.

Example 4: Meis1 Influences miR-146a Expression through Downregulation of PU.1

Next, the molecular mechanism by which Meis1 downregulates miR146a was investigated. No Meis1 binding site was found in the vicinity of the miR-146a locus in published Meis1 ChIP-seq profiles in myeloid cells (Heuser et al., 2011; Huang et al., 2012). However, miR-146a is known to be regulated by PU.1 (Spit) in macrophages (Ghani et al., 2011). Therefore, the binding of PU.1 to a previously identified PU.1 binding site located 10 kb upstream (−10 kb) of miR-146a was examined by ChIP-qPCR. This region exhibits epigenomic features of an active promoter, including an enrichment for H3K4me3 and binding of RNA Polymerase II in ENCODE data (Consortium, 2012). It was found that PU.1 binding to the —10 kb site was significantly reduced in H/M compared with H cells (FIG. 5A), suggesting that decreased PU.1 binding might be responsible for the downregulation of miR-146a. Consistent with this finding, lower PU.1 protein and mRNA levels in H/M compared with H cells (FIGS. 5B, 5C and 6F) were detected. In addition, an Integrated Motif Response Analysis (ISMARA) based on transcriptome profiles of H and H/M cells indicated decreased PU.1 activity in H/M relative to H cells.

Moreover, we found a significant association between low or no PU.1 protein expression and high expression of HOXA9 and MEIS1 (78.2% compared to 10% for HOXA9-overexpressing samples, p<9e-6, Fisher's exact test) in our AML patient cohort (FIGS. 5D and 5E). Finally, a 55% knockdown of PU.1 in H cells reduced miR-146a expression and increased Syk protein levels (FIGS. 5F and 5G). Taken together, these data indicate that by acting through PU.1, Meis1 indirectly influences the expression of miR-146a.

Example 5: Syk Overexpression Triggers a Meis1-Dependent Transcriptional Program

The inventors next sought to characterize the functional consequences of Syk overexpression in the context of Hoxa9-driven leukemias. For this purpose, the consequences of a lentiviral overexpression of human SYK (hSYK) in H cells in vitro and in vivo were examined. Of note, Syk expression levels were comparable between H/M cells and cells overexpressing Hoxa9 and hSYK (H/S). hSYK overexpression resulted in enhanced cell proliferation rates in the presence of IL3, IL6 and SCF, mimicking the overexpression of Meis1 (FIG. 6A). In addition, it enhanced the colony-formation capacity and replating efficiency of H cells in colony assays; both features suggest increased self-renewal. While Hoxa9 alone is sufficient to enable replating, both Meis1 and hSYK enhanced replating efficiency. This ability was significantly reduced by the Syk inhibitor R406, which decreased replating efficiency of H/M and H/S cells while moderately affecting colony formation and replating efficiency of H cells.

The inventors next investigated whether hSYK overexpression affected the leukemogenicity of H cells upon transplantation into lethally irradiated recipient mice. It was found that the combination of Hoxa9 and hSYK significantly increased the aggressiveness of the leukemias compared with Hoxa9 alone (median of 38.5 versus 103.5 days; p<0.001), with a median survival remarkably similar to that of Hoxa9/Meis1 (39 days; FIG. 6B). The observed leukemias were classified as AML with a dense infiltration of leukemic blasts in the bone marrow, spleen and liver, and leukemic blasts in the peripheral blood. Leukemias induced by the combination of Hoxa9 with hSYK or Hoxa9 with Meis1 were characterized by lower leukocyte counts and a more pronounced anemia compared to Hoxa9 alone. Immunophenotyping showed that the leukemic cells expressed c-Kit and the myeloid antigens Gr-1 and Mac-1, in agreement with previously published immunophenotypes of Hoxa9/Meis1-driven AML (Kroon et al., 1998). In addition, the frequency of c-Kit-positive cells was higher for Hoxa9 and Meis1, and for Hoxa9 and hSYK, compared to Hoxa9 alone, suggesting a more immature phenotype of the developing leukemias.

SYK activation depends on the phosphorylation of Y348 and Y352 (Kulathu et al., 2009). To test whether the accelerated leukemia development exhibited by H/S cells is dependent on SYK activation, H cells expressing either hSYK or a hSYK Y348F/Y352F double mutant were transplanted into lethally irradiated recipient mice and overall survival was monitored. Notably, hSYK double mutant abrogated the enhanced leukemogenicity of H/S cells, indicating that SYK activation is necessary for this feature.

The striking phenotypic similarity between H/M and H/S cells led the inventors to compare the transcriptional consequences of Meis1 and hSYK overexpression in Hoxa9 transformed myeloid progenitors by RNA-seq. By analyzing protein-coding and noncoding transcriptome compartments, it was found that both Meis1 and hSYK profoundly alter the transcriptome of H cells, leading to the differential expression of thousands of protein-coding genes and >100 long intergenic non-coding RNAs (lincRNAs; FIG. 6C). Intriguingly, these transcriptional changes were highly correlated (r=0.823) between H/M and H/S cells (FIG. 6D), which share a common transcriptional signature (FIG. 6E). Moreover, hSYK induced expression of Meis1 to levels comparable to those in H/M cells (FIG. 6F) and differentially expressed genes in H/M and H/S cells were similarly enriched for direct Meis1 binding to their promoter regions (Huang et al., 2012). Importantly, Meis1 expression is necessary for survival of H/S cells, as an inducible Meis1 knockout significantly affected H/S cell viability (FIG. 6G).

Together, these results indicate that Meis1 and Syk regulate highly overlapping transcriptional programs and implicate Meis1 as an effector of Syk signaling to chromatin.

Example 6: Hoxa9/Meis1-overexpressing myeloid progenitors are Syk-dependent

To test whether the enhanced Syk signaling observed in H/M cells could be exploited therapeutically, the effects of Syk inhibitors and of an shRNA-mediated knockdown of Syk in H/M cells, in vitro and in vivo were analyzed. As shown above, the Syk inhibitor R406 significantly reduced colony formation potential and replating efficiency in H/M cells.

The effect of Syk inhibition in H and H/M cells was further examined by monitoring the fate of individual cells and their progeny by time-lapse microscopy and single-cell tracking (Rieger et al., 2009). This allowed to track hundreds of H and H/M cells over more than 50 hours in real time and to record their history across cell generations. The analysis revealed a significant increase in cell death in R406 treated H/M cells compared with DMSO-treated cells, whereas H cells were not significantly affected. These results are not mediated by off-target effects of R406, as knocking down Syk in H/M cells with a doxycycline-inducible lentiviral shRNA resulted in decreased cell viability in vitro (FIGS. 7A and 7B). In addition, the inventors knocked down Syk in vivo by transplanting mice with cells that were either transduced with the doxycycline-inducible lentiviral Syk shRNAs or with control shRNAs and treating them with doxycycline for 43 days. Knockdown of Syk prolonged the survival of mice transplanted with H/M cells from a median time of 40.5 days in controls to a median of 103 days (p<0.001) (FIGS. 7C and 7D). Furthermore, mice transplanted with H/M or H cells were treated with the oral Syk inhibitor R788, a prodrug of R406. Seven days of treatment with R788 reduced the percentage of leukemic cells in mice transplanted with H/M cells by more than 70% on average, while barely affecting the level of H cells (FIG. 7E). Treatment with R788 for 20 days significantly prolonged the survival of mice transplanted with H/M cells from a median of 38 days to a median of 130 days (p<0.001; FIG. 7F). No significant effect was observed in mice transplanted with H cells.

Given the pronounced sensitivity of Hoxa9/Meis1-transformed mouse hematopoietic progenitors to Syk inhibition, it was examined whether this effect can also be recapitulated in primary human AML samples. For this purpose, three AML samples exhibiting strong HOXA9 expression and weak MEIS1 expression were considered, and compared to three samples expressing both genes at high levels (FIG. 7G). Notably, none of these samples harbored activating mutations in FLT3. It was found that AML samples expressing high levels of both HOXA9 and MEIS1 exhibited increased expression of SYK and pSYK, and were more sensitive to the SYK inhibitors PRT062607 and R406 compared to samples with weak MEIS1 expression (FIGS. 7H-I). Importantly, Syk inhibition did not affect the viability of CD34+ progenitor cells isolated from healthy donors (FIG. 7J). Finally, Syk inhibition significantly prolonged survival of NSG mice transplanted with patient-derived AML cells overexpressing HOXA9 and MEIS1, with no significant difference for HOXA9 alone (FIG. 7K).

In summary, these results demonstrate that enhanced Syk signaling in the presence of Meis1 represents a regulatory feedback mechanism of leukemogenesis in Hoxa9driven AML that renders these cells sensitive to Syk inhibition.

Several studies characterized gene expression signatures and individual target genes regulated by Hoxa9 and Meis1. Among those, only a few at most partially recapitulate the oncogenic effects of Hoxa9 and Meis1.

In this work, the inventors employed quantitative mass spectrometry to study proteomic and phosphoproteomic changes induced by Meis1 overexpression, identify Meis1 regulated proteins and signaling pathways, and investigated their therapeutic potential. By this approach upregulation and activation of Syk by Meis1 as a key leukemogenic mechanism in a Hoxa9-driven mouse model system and in a subset of human AMLs was identified.

Syk was originally described as a signaling mediator downstream of the B-cell antigen receptor, but it has also been identified as a drug target for the treatment of AML (Hahn et al., 2009). In addition, Syk has been shown to be activated by integrin signaling, to phosphorylate STAT5 in AML (Miller et al., 2013; Oellerich et al., 2013) and to cooperate with FLT3-ITD during the induction and maintenance of myeloid leukemias (Puissant et al., 2014). Moreover, SYK Y323 phosphorylation in AML has recently been correlated with an unfavourable prognosis (Boros et al., 2015) and activatory SYK phosphorylation (pY348) correlates with poor event-free and re-lapsefree survival in a AML patient cohort.

The results indicate that Syk protein levels, but not mRNA levels, are upregulated upon overexpression of Meis1 through a post-transcriptional mechanism. By analyzing Meis1-dependent miRNA expression changes, the inventors found that Meis1 downregulates miR-146a, which in turn regulates Syk expression posttranscriptionally. The invention has thereby identified a previously unrecognized regulatory link between miR-146a and Syk that is indirectly orchestrated by Meis1.

Our results implicate Syk in Meis1-mediated leukemic transformation. Syk potently cooperates with Hoxa9 for leukemic transformation and is strikingly similar to Meis1 with regard to its leukemogenic potential. This similarity is furthermore underscored by the ability of Syk to induce a Meis1 transcriptional program in the context of Hoxa9 overexpression. Notably, Syk does not render Hoxa9-transformed cells independent of Meis1, indicating a cell-intrinsic dependency.

Meis1 enhances Syk expression through a regulatory feedback circuit. In addition, Meis1 also upregulates the Syk activator Itgb3 and downregulates the phosphatase Ptpn6 (log fold change =−0.345, adj p=2.88e-17; H/M vs H cells), a known negative regulator of Syk activity. Additional signaling effectors that might contribute to Syk activation in our circuit remain to be identified.

The largely overlapping transcriptional consequences of Syk and Meis1 led the inventors to hypothesize that Meis1-transformed leukemias would be more addicted to Syk than to other signaling proteins such as Flt3, which has previously been shown to be dispensable for Meis1-driven leukemias (Morgado et al., 2007). The orthogonal treatment results of the invention, based on both shRNA-mediated knockdown and pharmacological inhibition of Syk, showed that Hoxa9/Meis1-transformed leukemias were clearly more sensitive to Syk inhibition than Hoxa9-transformed leukemias.

In summary, the invention provides a Meis1-dependent feedback loop involving PU.1, miR-146a and Syk that promotes cell survival and can be targeted by Syk inhibitors.

Experimental Procedures

Vectors

Retroviral vectors MSCV-Meis1-IRES-YFP (Pineault et al., 2003) and MSCV-Hoxa9PGK-neo (Kroon et al., 1998) and their respective control vectors have been described previously.

Mice and retroviral infection of lineage-depleted bone-marrow cells C57BL/6J mice for transplantation experiments were obtained from Janvier-Labs (Le Genest-Saint-Isle, France), B6/miR-146a−/−mice (Boldin et al., 2011) were purchased from the Jackson Laboratory (Bar Harbor, USA) and maintained at the Zentrale Forschungseinrichtung (ZFE) of the Goethe University of Frankfurt. Bone marrow cells were harvested from mice, and lineage-negative cells were obtained by negative selection using a Lineage Cell Depletion Kit (mouse) (Miltenyi Biotec, Bergisch Gladbach, Germany) following the manufacturer's instructions. Lineagenegative cells derived from C57BL/6J were retrovirally infected by co-culture with GP+E86 cells in the presence of polybrene (10 μg/ml, Sigma-Aldrich, Munich, Germany) .

Mass Spectrometry and Data Analysis

Mass spectrometry and data analysis were performed essentially as described in (Corso et al., 2016). A detailed description is provided in the Supplemental Experimental Procedures.

Accession Numbers

Mass spectrometry data have been deposited to the PRIDE Archive (project number PXD004192; username: reviewer39570@ebi.ac.uk; password: YVdoHZ8T). RNAseq data have been deposited to the Short Read Archive under accession number PRJNA322136. miRNA microarray data have been deposited under GEO accession number GSE74566.

1-19. (canceled)

20. A method selected from:
A) determining the sensitivity of a cancer patient for a Spleen Tyrosine Kinase (SYK) inhibitor therapy, the method comprising the steps of
(a) Determining in a biological sample of the patient the amount or level of
(i) Meis1 and Hoxa9, and/or
(ii) miR-146a, and/or
(iii) PU.1
(b) Comparing the amount or level as determined in (a) with a reference or control,
Wherein an increased amount or level compared to the reference or control of (i), or a reduced amount or level compared to the reference or control of (ii) and/or (iii), indicates sensitivity of the patient for a SYK inhibitor therapy;
B) diagnosing a SYK inhibitor treatable cancer disease in a subject, the method comprising:
(a) determining in a biological sample of the subject the amount or level of Meis1, or Meis1 and Hoxa9 expression,
(b) comparing the amount or level of Meis1, or Meis1 and Hoxa9 expression, as determined in (a), with a control or reference,
wherein an increased amount or level of Meis1, or Meis1 and Hoxa9 expression in the biological sample compared to the control or reference, indicates the presence of a SYK inhibitor treatable cancer disease in a subject;
C) diagnosing a SYK inhibitor treatable cancer disease in a subject, the method comprising:
(a) determining in a biological sample of the subject the amount or level of PU.1 expression,
(b) comparing the amount or level of PU.1 expression, as determined in (a), with a control or reference,
wherein a decreased amount or level of PU.1 expression in the biological sample compared to the control or reference, indicates the presence of a SYK inhibitor treatable cancer disease in a subject: and
D) diagnosing a SYK inhibitor treatable cancer disease in a subject, the method comprising:
(a) determining in a biological sample of the subject the amount or level of miR-146a expression,
(b) comparing the amount or level of miR-146a expression, as determined in (a), with a control or reference,
wherein a decreased amount or level of miR-146a expression in the biological sample compared to the control or reference, indicates the presence of a SYK inhibitor treatable cancer disease in a subject.

21. The method according to claim 20, wherein the cancer is leukemia.

22. The method according to claim 20, wherein the cancer is a Hox expression driven cancer.

23. The method according to claim 20, wherein in (i) the mRNA expression of Meis1 and Hoxa9 mRNA is determined, and/or, in (iii) the miRNA expression of miR-146a is determined, and/or in (iii) the PU.1 protein expression and/or mRNA expression is determined.

24. The method according to claim 20, wherein the biological sample of the patient is a blood sample or bone marrow sample.

25. The method according to claim 20, wherein the method is an in vitro or an ex vivo method.

26. The method according to claim 20, wherein the patient is a relapsed cancer patient and/or a cancer patient who received one or more non successful cancer therapies.

27. A method for treating a cancer disease in a patient, the method comprising the steps of
(a) obtaining a biological sample from the patient,
(b) performing a method according to claim 20,
(c) administering to the patient a therapeutically effective amount of a cancer therapeutic,
wherein the cancer therapeutic is selected from a SYK inhibitor if an increased amount or level compared to the reference or control of (i), or a reduced amount or level compared to the reference or control of (ii) and/or (iii), is determined in the biological sample of the patient; or wherein the cancer therapeutic is selected from a compound other than a SYK inhibitor if no increased amount or level compared to the reference or control of (i), and no reduced amount or level compared to the reference or control of (ii) and/or (iii) is determined.

28. The method according to claim 27, wherein the cancer disease is leukemia.

29. The method according to claim 27, wherein the patient is a human.

30. The method according to claim 20, wherein the SYK inhibitor is a small molecule inhibitor, an antibody and/or an expression modulator selected from antisense molecules, microRNAs, siRNAs, and aptamers.

31. The method according to claim 20, wherein mRNA expression and/or miR expression is determined by a method selected from PCR-based methods or in situ hybridization techniques, and/or wherein protein expression is determined by using an antibody based detection assay.

32. A diagnostic kit for use in performing a method according to claim 20, comprising components for the detection of the amount or level of any of the biomarkers selected from Meis1, Hoxa9, miR-146a and PU.1.

33. The diagnostic kit according to claim 32, comprising components for the detection of the amount or level of Meis1 and Hoxa9, optionally for miR-146a and/or PU.1

34. The diagnostic kit according to claim 32, comprising components for the detection of the amount or level of miR-146a and/or PU.1.

35. The diagnostic kit according to claim 32, further comprising a SYK inhibitor in an amount that, when administrated to a cancer patient, is effective for the treatment of the cancer.

36. The method, according to claim 21, wherein the leukemia is acute myeloid leukemia (AML).

37. The method, according to claim 20, wherein the cancer is Hoxa9 driven AML.

38. The method, according to claim 27, wherein the cancer is Hoxa 9 driven AML.

39. The method according to claim 30, wherein the SYK inhibitor is entospletinib, ibrutinib, idelalisib, fostamatinib (R788), R406 or piceatannol.
